(19) United States
(12) Patent Application Publication
(10) Pub. No.: US 2009/0060959 A1
(43) Pub. Date: Mar. 5, 2009

(54) HYDROUS POWDERY COSMETIC PREPARATION

(75) Inventor: Keiji Igarashi, Tokyo (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Kose Corporation, Chuo-ku, Tokyo (JP)

(21) Appl. No.: 11/909,657

(22) PCT Filed: Mar. 7, 2006
(86) PCT No.: PCT/JP2006/304300

§ 371 Date: Sep. 25, 2007

(30) Foreign Application Priority Data
Mar. 25, 2005 (JP) 2005-089898

(51) Int. Cl. A61K 8/02

(52) U.S. Cl. 424/401

(57) ABSTRACT

A water-containing powdery cosmetic preparation comprises (a) hydrophobic particles having a surface area of at least 80 m²/g, (b) plate-like particles comprising a polymer obtained by polymerizing or copolymerizing one or more types of monomers selected from the group consisting of styrenes, methacrylates, and acrylates, and (c) an aqueous component, wherein the water-containing powdery composition is liquefied by application with rubbing. The water-containing powdery cosmetic preparation has good storage stability, imparts an excellent fresh feeling upon use without a powdery or frictional feel, exhibits uniform applicability without caking or streaking, and exhibits excellent cosmetic retention without darkening over time.

### TECHNICAL FIELD

The present invention relates to a hydrous (water-containing) powdery cosmetic preparation comprising an aqueous component covered with hydrophobic particles as a water-containing powdery composition, wherein the water-containing powdery composition breaks up and is liquefied when the water-containing powdery cosmetic preparation is applied to and rubbed on the skin. More particularly, the present invention relates to a water-containing powdery cosmetic preparation which imparts a fresh feeling free of a powdery or frictional feel, can be uniformly applied to the skin with rubbing using the fingers, hand, or a small tool without streaking or caking, has excellent retention over time particularly when a color material is added, and has good storage stability.

### BACKGROUND ART

Water-containing powdery cosmetic preparations are in the form of a powder before use and are liquefied at the time of use by applying to the skin with rubbing to give properties similar to those of a face lotion or a milky lotion. Such water-containing powdery cosmetic preparations comprise a water-containing powdery composition in which an aqueous component is covered with hydrophobic particles (refer to Patent Document 1). Since the aqueous component of the water-containing powdery composition is covered with the hydrophobic particles, the water-containing powdery composition has the appearance of a powder and, when used, the hydrophobic particles touch the skin and then the inner aqueous component is materialized by rubbing. Thus, a frictional and powdery feeling occurs between the time that sufficient aqueous component is evident and a freshness and a cool feeling are sensed.

Many attempts have been made to solve such a problem. For example, a cosmetic preparation in which an aqueous gel is used as the aqueous component for the purposes of improving the production stability and the storage stability of the water-containing powdery composition (refer to Patent Document 2), a highly transparent powdery cosmetic preparation in which a large amount of powdery component such as a pearlescent agent or a lamellar agent is added to prevent streaking at the time of application (refer to Patent Document 3), and the like have been developed.

### Patent Document 1: JP-A-5-65212

### Patent Document 2: JP-A-2001-131528

### Patent Document 3: JP-A-2003-267826

### DISCLOSURE OF THE INVENTION

### Problems to be Solved by the Invention

However, when the powdery cosmetic preparation in which the aqueous component in the water-containing powdery composition is gelled is applied to the skin using the fingers, hand, or a small tool, caking or streaking easily occurs so that uniform application may become difficult. In the water-containing powdery cosmetic preparation including a large amount of powdery component, although streaking at the time of application can be prevented and excellent transparency is obtained, cosmetic retention when a color material is added and storage stability may be inferior. Thus, development of a water-containing powdery cosmetic preparation is desired which does not lose the fresh feeling upon use, can be uniformly applied to the skin with rubbing using the fingers, hand, or a small tool without caking or streaking, has excellent cosmetic retention which prevents darkening over time when a color material is added, and has very good storage stability.

### Means for Solving the Problems

As a result of extensive research in consideration of the above-mentioned situation, the inventor has found that, if specific hydrophobic particles, specific plate-like particles, and an aqueous component are combined, a water-containing powdery cosmetic preparation can be obtained having good storage stability, excellent applicability, an excellent fresh feeling free of a frictional feel, and excellent cosmetic retention which prevents darkening over time particularly when a color material is added. Furthermore, the inventor has found that storage stability can be further improved by gelling the aqueous component.

The present invention was completed based on the above-mentioned finding, and provides a water-containing powdery cosmetic preparation comprising:

(a) 0.1 to 7% by mass of hydrophobic particles having a surface area of at least 80 m²/g;
(b) 0.5 to 30% by mass of plate-like particles comprising a polymer obtained by polymerizing or copolymerizing one or more types of monomers selected from the group consisting of styrenes, methacrylates, and acrylates; and
(c) 50 to 90% by mass of an aqueous component;

wherein the water-containing powdery composition is liquefied by application with rubbing.

The present invention also provides the above water-containing powdery cosmetic preparation, wherein the component (c) comprises an aqueous gelling agent so as to form an aqueous gel.

The present invention further provides the above powdery cosmetic preparation comprising (d) 0.3 to 5% by mass of an oily component.

### EFFECT OF THE INVENTION

The water-containing powdery cosmetic preparation of the present invention has good storage stability, an excellent fresh feel upon use free of a powdery or frictional feel, and excellent applicability which produces a uniform cosmetic film without caking or streaking, and excellent cosmetic retention which prevents darkening over time particularly when a color material is added.

### BEST MODE FOR CARRYING OUT THE INVENTION

The hydrophobic particles having a surface area of at least 80 m²/g, which are the component (a) in the water-containing powdery cosmetic preparation of the present invention, are not limited insofar as the hydrophobic particles have the above surface area and have a surface that shows hydrophobicity. As examples thereof, particles which are themselves hydrophobic, particles obtained by surface-treating hydrophilic particles with a well-known hydrophobizing agent, and particles which are obtained by further surface-treating hydrophobic particles with a hydrophobizing agent in order to increase the degree of hydrophobicity can be given. Whether particles are hydrophobic or not is determined by dispersing the particles in water. Hydrophobic particles are particles which do not settle or disperse but float on the water even after stirring.

Regarding the component (a), as examples of hydrophobic particles which are themselves hydrophobic, organic resin powders such as polystyrene powder, polyethylene powder, organopolysiloxane powder, N-acyl-lysine powder, polyethylene tetrafluoride powder, and epoxy resin powder and metallic soap powders such as aluminium stearate, zinc laurate, and magnesium stearate can be given.

As examples of hydrophilic particles hydrophobized by a surface treatment, inorganic particles such as titanium oxide, zinc oxide, silcic acid anhydride, aluminum oxide, magnesium oxide, zirconium oxide, magnesium carbonate, calcium carbonate, aluminum silicate, magnesium silicate, aluminum magnesium silicate, mica, synthetic mica, synthetic sericite, sericite, talc, silicon carbide, barium sulfate, boron nitride, bismuth oxychloride, and mica titanium; organic particles such as silk powder, starch, nylon, and crystalline cellulose; and composite particles such as mica titanium coated with particulate titanium oxide, mica titanium coated with particulate zinc oxide, or mica titanium coated with barium sulfate can be given.

As examples of hydrophobizing agents used to hydrophobize the particles, organosilicon compounds such as various trimethyl silylation agents and methyl hydrogen polysiloxane; fluorine compounds such as perfluoroalkyl phosphate, perfluoropolyether phosphate, and perfluoroalkylsilane; metallic soaps; and oily agents can be given. Among these, organosilicon compounds and fluorine compounds are preferable since they further improve the degree of hydrophobicity.

As the hydrophobic particles, which are the component (a), fumed silicic acid anhydride which has an average particle size of 0.001 to 0.1 μm and which is treated with a hydrophobizing agent is particularly preferable from the point of storage stability of the water-containing powdery cosmetic composition. Specifically, the commercially available products AEROSIL R974, AEROSIL R972, AEROSIL RX200, and AEROSIL RX300 (all manufactured by Nippon Aerosil Co., Ltd.) and CAB-O-SIL TS-530 (manufactured by Cabot Corporation) can be given as examples.

The amount of the component (a) used in the present invention is 0.1 to 7% by mass (hereinafter shown simply as “%”), and particularly preferably 2 to 5% of the total amount of the water-containing powdery cosmetic composition. If the amount of the component (a) is less than 0.1%, the water of the component (c) cannot be covered with the component (a). If the amount of the component (a) is more than 7%, liquefaction at the time of use becomes difficult. If the surface area is less than 80 m²/g, the aqueous component cannot be cleanly coated and thus, powderization is not possible.

The plate-like particles, which are the component (b) in the powdery cosmetic preparation of the present invention, are a polymer obtained by polymerizing or copolymerizing one or more types of monomers selected from the group consisting of styrenes, methacrylates, and acrylates.

As the component (b), plate-like particles having a thickness of preferably 0.1 to 20 μm, and more preferably of 0.5 to 2 μm, an average particle size of preferably 5 to 60 μm, and more preferably 15 to 35 μm, and an aspect ratio of at least 10 are preferable. By using particles within these ranges, a good water-containing powdery cosmetic preparation having a soft feel, good slidability, adhesion to the skin, no darkening over time, and retention of the transparent feel of the applied film can be obtained.

The component (b) has a bulk specific gravity of preferably 5 to 20 ml/g, and particularly preferably 10 to 17 ml/g. The bulk specific gravity may be measured by JISK-5101 (standing method). If plate-like particles having a bulk specific gravity within these ranges are used, dispersibility in the cosmetic preparation is excellent and a cosmetic preparation having uniform applicability without a powdery or frictional feel and without streaking or caking can be obtained.

As the preparation method of the component (b), the method disclosed in JP-A-2004-2274 can be given, for example. As an example of a particularly preferable method, a method can be given which includes developing a polymer solution obtained by dissolving a polymer in an organic solvent on a liquid layer which is liquid at room temperature, removing the solvent to form a thin film, and pulverizing the thin film. According to this method, particles having the smoothness of the solution surface of the solution layer and having a uniform thickness are obtained and when included in the water-containing powdery cosmetic preparation, a good cosmetic preparation having excellent tactile sensation on use such as feel, slidability, and a sense of adherence, no darkening of the cosmetic film over time, and retention of transparency can be obtained.

As the plate-like particles, which are the component (b), particles which have been surface-treated using a fluorine compound, a silicone compound, a metallic soap, a wax, an oil or fat, or a hydrocarbon may be used. These may be used alone or in combination of two or more.

The amount of the component (b) used in the present invention is preferably 0.5 to 30%, and particularly preferably 3 to 15% of the total amount of the water-containing powdery cosmetic preparation. If the amount of the component (b) is less than 0.5%, the cosmetic film darkens over time. If the amount of the component (b) is more than 30%, the powdery property increases and a fresh feeling upon use cannot be obtained.

As the aqueous component (component (c)) used in the water-containing powdery cosmetic preparation of the present invention, water or any compound soluble in water can be used without specific limitations. Examples include, in addition to water, glycols such as propylene glycol, 1,3-butylene glycol, dipropylene glycol, and polyethylene glycol; glycerols such as glycerol, diglycerol, and polyglycerol; and extracts from plants such as aloe vera, hamamelis, cucumber, lemon, lavender, and rose. These aqueous components may be used either alone or in combination of two or more.

In the present invention, an aqueous gel can be formed by adding an aqueous gelling agent to the component (c). It is preferable to prepare the water-containing powdery cosmetic preparation by covering the aqueous gel with hydrophobic particles or the like.

The aqueous gelling agent is dissolved or swelled in water and gels the water. As examples, naturally occurring plant polymers such as agar, guar gum, locust bean gum, quince seed gum, furcellaran, carageenan, sodium alginate, gellan gum, starch, pectin, and konjak powder; naturally occurring microorganism-based polymers such as sclerotium gum; naturally occurring polymers of animal origin such as gelatin; half-synthesized polymers such as cellulose derivatives, for example, methylcellulose, hydroxymethylcellulose, hydroxyethylcellulose, hydroxypropylcellulose, carboxymethylcellulose; carboxyvinyl (co)polymers such as polyvinyl alcohol, polyvinyl pyrrolidone, and vinyl acetate (co)polymers; hydrophilic acrylic polymers such as (meth)acrylic acid (co)polymers and sodium (meth)acrylate (co)polymers; and water swelling clay minerals such as magnesium sodium silicate, hydrous aluminium silicate, montmorillonite, saponite, hectolite, silicon mica tetraflouride; and the like can be given. These may be used individually or in combination of two or more. Among these aqueous gelling agents, agar, gelatin, carageenan, gellan gum, magnesium sodium silicate, sclerotium gum, and the like are preferable from the viewpoint of storage stability.

The aqueous gelling agent is added in an amount sufficient to gel the component (c). Although the specific amount varies depending on the type of the aqueous gelling agent, 0.1 to 10% of the total amount of the component (c) is preferable. When the aqueous gelling agent requires alkalinity in order to gel, an alkaline compound such as sodium hydroxide, potassium hydroxide, ammonium hydroxide, triethylamine, triethanolamine, diethanolamine, and the like may be used. In this instance, depending on the type of aqueous gelling agent used, the ratio of the aqueous gelling agent and the alkali is preferably 1:0.001 to 1:1.

The component (c) of the water-containing powdery cosmetic preparation is preferably used in an amount of 50 to 90%, and particularly preferably 65 to 85% of the total amount of the water-containing powdery cosmetic preparation. If more than 90%, it is difficult for the cosmetic preparation to exhibit makeup retention. If less than 50%, it is difficult for the cosmetic preparation to impart a fresh feeling upon use.

An oily component may further be added to the water-containing powdery cosmetic preparation of the present invention as the component (d). As the component (d), any oily component, irrespective of origin (animal oils, vegetable oils, and synthetic oils) and properties (solid oils, half-solid oils, liquid oils, and volatile oils), such as hydrocarbons, oils and fats, waxes, hydrogenated oils, ester oils, fatty acids, higher alcohols, silicone oils, fluorine-containing oils, lanolin derivatives, oily gelling agents, oleophilic surfactants, and oil-soluble UV absorbers can be used.

Specific examples include hydrocarbons such as liquid paraffin, squalane, petroleum jelly, polyisobutylene, polybutene, paraffin wax, ceresin wax, microcrystalline wax, montan wax, and Fischer-Tropsch wax; oils and fats such as olive oil, castor oil, mink oil, and macadamia nut oil; waxes such as bees wax, carnauba wax, Candelilla wax, and spermaceti; esters such as cetyl isooctanate, isopropyl myristate, isopropyl palmitate, octyldodecyl myristate, glyceryl trioctanoate, polyglyceryl diisostearate, diglyceryl triisostearate, glyceryl tribehenate, pentaerythritol rosinate, neopentyl glycol dioctanoate, cholesterol fatty acid ester, and di(cholesteryl/behenyl/octyldodecyl)N-lauroyl-L-glutamate; fatty acids such as stearic acid, lauric acid, myristic acid, behenic acid, isostearic acid, oleic acid, colophony acid, and 12-hydroxystearic acid; higher alcohols such as stearyl alcohol, cetyl alcohol, lauryl alcohol, oleyl alcohol, isostearyl alcohol, and behenyl alcohol; silicones such as low molecular weight dimethylpolysiloxane, high molecular weight dimethylpolysiloxane, methylphenylpolysiloxane, decamethylcyclopentasiloxane, octamethylcyclotetrasiloxane, polyether-modified polysiloxane, cross-linked organopolysiloxane, and fluorine-modified silicone; fluorine-containing oils such as perfluoropolyether, perfluorodecane, and perfluorooctane; lanolin derivatives such as lanolin, lanolin acetate, lanolin fatty acid isopropyl, and lanolin alcohol; oleophilic surfactants such as sorbitan sesquioleate; oily gelling agents such as dextrin fatty acid ester, sucrose fatty acid ester, starch fatty acid ester, 12-hydroxystearic acid, and calcium stearate; oleophilic surfactants such as glycerol monostearate and sorbitan tristearate; and oil-soluble UV absorbers such as p-ethylaminobenzoate, 2-ethylhexyl p-methoxycinnamate, 4-tert-butyl-4′-methoxydibenzoylmethane, and oxybenzone can be given. These oily components may be used either individually or in combination of two or more.

In order to incorporate the component (d) in the cosmetic preparation, finely powderized high-melting point waxes such as synthetic hydrocarbon waxes; powders obtained by causing an oily component to be adsorbed in and supported on porous particles such as particles of magnesium carbonate and highly agglomerated polymers such as acrylate copolymers, and powderizing such porous particles; capsules made from an oily component such as polymethyl methacrylate; and the like can be used. The oily component may further be added in the form of an oil-in-water emulsion prepared by dispersing the component (d) in water which is used as the aqueous component of the component (c).

The amount of the component (d) is preferably 0.3 to 5%, and particularly preferably 0.5 to 3% of the total amount of the water-containing powdery cosmetic preparation. The addition of the component (d) in these ranges ensures production of a powdery cosmetic preparation which can excellently attach to the skin and exhibits excellent makeup retention and storage stability.

In addition to the above components, various additives commonly used in cosmetic preparations and quasi-drug preparations can be added to the water-containing powdery cosmetic preparation of the present invention to the extent the effect of the present invention is not adversely affected. Such additives include powders other than the component (a) and the component (b), hydrophilic surfactants, fresheners such as an alcohol and menthol, film-forming agents, hydrophilic UV absorbers, salts, pH adjusting agents, antioxidants, humectants, cosmetic components such as vitamin C derivatives, antiseptic agents such as p-hydroxy benzoate esters and phenoxy ethanol, and perfumes. These additives can be added as appropriate to impart various effects.

Among these additives, as the powder other than the component (a) or the component (b), inorganic powders, organic powders, coloring material powders, and composite material powders, and the like can be used without particular limitations to the shape (including spherical, acicular, plate-like, and spindle-shaped), particle structure (including porous and non-porous), and particle size (including fog-like, fine particles, and pigment-class) as a base component, a component to adjust feel, a coloring component, and the like. The surface of these particles may be treated with a fluorine compound, a silicone compound, a metallic soap, a wax, a fat, an oil, a hydrocarbon, or the like.

Although there are no particular limitations, the water-containing powdery cosmetic preparation can be prepared by, for example, a method of mixing the components (a) and (c), and blending the resulting mixture with the component (b), or a method of powderizing the component (b) after mixing with the component (d), as required, and adding the resulting mixture to a mixture of the component (a) and the component (c), followed by powderization.

As another preferable method for preparing the water-containing powdery cosmetic preparation of the present invention, a method of preparing a water-containing powdery composition by covering the below-described aqueous gel particles, used as the component (c), with the component (a), adding the component (b) and other optional components to the resulting particles, and blending the resulting mixture can be given. The water-containing powdery composition obtained in this manner is preferable due to its storage stability.

From the viewpoint of excellent storage stability, the aqueous gel used as the component (c) in this method is preferably used in a state in which the aqueous gel is hardened in the shape of particles. Although not particularly limited, a method of preparing an aqueous gel from an aqueous phase and an aqueous gelling agent, heating the resulting aqueous gel, and shearing the gel at a high speed, a method of freeze-shattering, and the like can be given as the method for preparing the aqueous gel particles. As the method of high speed shearing, use of a high speed pin mill, a cutter-mixer, and the like can be given. As the method of freeze shattering, freezing of the aqueous gel using a refrigerant such as liquid nitrogen and then pulverizing the aqueous gel can be given. The average particle size of the aqueous gel particles is preferably between 1 μm and 300 μm.

There are no particular limitations to the method of covering the surface of the aqueous gel particles with the hydrophobic particles. One example of such a method comprises placing the aqueous gel particles of the component (c) in a stirring vessel and adding the hydrophobic particles of the component (a) while stirring at a low temperature to prevent fusion or aggregation due to a rise of temperature. The stirring vessel used preferably has a jacket with a cooling mechanism and stirring blades that rarely come in contact with the wall and bottom of the jacket.

In preparing the water-containing powdery composition, the particle size of the hydrophobic particles of the component (a) must be smaller than the particle size of the aqueous gel particles of the component (c). The particle size of the hydrophobic particles (component (a)) is preferably 1/10 or less of the particle size of the aqueous gel particles (component (c)) when effect of covering the aqueous gel particles is considered. The ratio of the aqueous gel particles and the hydrophobic particles is preferably 100:0.5 to 100:25, although the particular ratio varies according to the particle size of the aqueous gel particles and the hydrophobic particles.

The water-containing powdery cosmetic preparation of the present invention obtained in this manner can be used as a makeup cosmetic such as an eye color, foundation, blusher, and face powder; a skin care cosmetic such as a whitening powder, a body powder, an anti-perspirant powder, a face lotion, and a milky lotion; and a sunburn preventive.

### EXAMPLES

The present invention will be explained in more detail by examples which are not intended to be limiting of the present invention.

### Example 1

### Water-Containing Powdery Face Powder

Water-containing powdery face powder of Examples 1 to 6 (Present Invention Preparations 1 to 6) and Comparative Examples 1 to 5 (Comparative Preparations 1 to 5) were prepared using the components shown in Table 1. The resulting water-containing powdery face powders were evaluated for feeling upon use (freshness), uniform applicability (no streaking or caking), makeup retention (no darkening with time, retention of transparent feel), and storage stability. The results are also shown in Table 1.

TABLE 1

| | | | | | | | | | | | | (%) |
|---|---|---|---|---|---|---|---|---|---|---|---|---|
| | | Present Invention Preparation | | | | | | Comparative Preparation | | | | |
| No. | Component | 1 | 2 | 3 | 4 | 5 | 6 | 1 | 2 | 3 | 4 | 5 |
| 1 | Agar | 1.5 | 1.5 | — | 1.5 | 1.5 | — | 1.5 | 1.5 | 1.5 | 1.5 | 1.5 |
| 2 | Locust bean gum | 0.1 | 0.1 | — | 0.1 | 0.1 | — | 0.1 | 0.1 | 0.1 | 0.1 | 0.1 |
| 3 | Carrageenan | 0.05 | 0.05 | — | 0.05 | 0.05 | — | 0.05 | 0.05 | 0.05 | 0.05 | 0.05 |
| 4 | Glycerin | 1 | 1 | 1 | 1 | 1 | 1 | 1 | 1 | 1 | 1 | 1 |
| 5 | 1,3-Butylene glycol | 10 | 10 | 10 | 10 | 10 | 10 | 10 | 10 | 10 | 10 | 10 |
| 6 | Methyl p-hydroxybenzoate | 0.15 | 0.15 | 0.15 | 0.15 | 0.15 | 0.15 | 0.15 | 0.15 | 0.15 | 0.15 | 0.15 |
| 7 | Purified water | 68.4 | 68.9 | 70.05 | 53.9 | 75.4 | 68.55 | 71.39 | 61.4 | 25.9 | 75.9 | 68.4 |
| 8 | Hydrophobized silicic anhydride*¹ | 3 | 3 | 3 | 0.5 | 3 | 5 | 0.01 | 10 | 3 | 3 | 3 |
| 9 | Plate-like polymethylmethacrylate*² | 7.5 | 7.5 | 7.5 | 25 | 1 | 7.5 | 7.5 | 7.5 | 50 | — | — |
| 10 | Spherical polymethylmethacrylate | — | — | — | — | — | — | — | — | — | — | 7.5 |
| | (average size: 10 μm) | | | | | | | | | | | |
| 11 | Silicone-treated titanium oxide*³ | 1 | 1 | 1 | 1 | 1 | 1 | 1 | 1 | 1 | 1 | |
| 12 | Colcothar | 0.2 | 0.2 | 0.2 | 0.2 | 0.2 | 0.2 | 0.2 | 0.2 | 0.2 | 0.2 | 0.2 |
| 13 | Yellow iron oxide | 0.6 | 0.6 | 0.6 | 0.6 | 0.6 | 0.6 | 0.6 | 0.6 | 0.6 | 0.6 | 0.6 |
| 14 | Fluorine-treated talc*⁴ | 3 | 3 | 3 | 3 | 3 | 3 | 3 | 3 | 3 | 3 | 3 |
| 15 | Metallic soap-treated sericite*⁵ | 2 | 2 | 2 | 2 | 2 | 2 | 2 | 2 | 2 | 2 | 2 |
| 16 | Calcium carbonate | 1 | 1 | 1 | 1 | 1 | 1 | 1 | 1 | 1 | 1 | 1 |
| 17 | Dimethyl polysiloxane*⁶ | 0.5 | — | 0.5 | — | — | — | 0.5 | 0.5 | 0.5 | 0.5 | 0.5 |
| Evaluation item and result | | | | | | | | | | | | |
| a | Freshness | AAA | AAA | AAA | BBB | AAA | BBB | AAA | DDD | DDD | AAA | AAA |
| b | Uniform applicability | AAA | AAA | AAA | AAA | BBB | AAA | BBB | CCC | CCC | DDD | DDD |
| | (no streaking/caking) | | | | | | | | | | | |
| c | Makeup retention | AAA | AAA | AAA | AAA | BBB | AAA | AAA | CCC | CCC | CCC | CCC |
| d | Storage stability | AAA | AAA | AAA | AAA | AAA | AAA | DDD | AAA | BBB | AAA | BBB |

*¹AEROSIL RX-300 (manufactured by Nippon Aerosil Co., Ltd.)
*²Plate-like particles obtained by pulverizing polymethylmethacrylate into a thin film (average particle size: 20 μm, average particle thickness: 1 μm, bulk specific gravity: 15 ml/g)
*³Treated with 3% methylhydrogen polysiloxane
*⁴Treated with a 5% perfluoroalkyl-phosphate diethanolamine salt
*⁵Treated with 3% zinc laurate
*⁶KF-96 10CS (manufactured by Shin-Etsu Chemical Co.. Ltd.)

### (Method of Preparation)

A. Components 1 to 7 were heated and melted at 85° C., cooled to solidify, and pulverized at −80° C. to obtain an aqueous gel particle (30 to 100 μm). With respect to Present Invention 3 and 6, aqueous products were obtained by heating and melting the components 4 to 7.
B. A component 8 was added to A and the mixture was blended using a Henschel mixer (manufactured by Mitsui Miike Machinery Co., Ltd.) to obtain water-containing powdery compositions.
C. Previously blended components 9 to 17 were added to B and the mixture was blended using a Henschel mixer to obtain water-containing powdery face powders.

### (Evaluation Method 1)

The following items (a) to (c) were evaluated for each sample by 20 expert panelists, who scored each sample 0 to 6 points according to the following evaluation standard. An average of the scores given by all panelists was calculated for each sample. Based on the average score, the samples were judged according to the following criterion in four grades.

### Evaluation Item:

(a) Feeling upon use (freshness)
(b) Uniform applicability (no streaking or caking)
(c) Makeup retention (no darkening with time, retention of transparent feel)

### Evaluation Standard:

| (Score) | (Evaluation) |
|---|---|
| 6 | Excellent |
| 5 | Good |
| 4 | Slightly good |
| 3 | Fair |
| 2 | Slightly bad |
| 1 | Bad |
| 0 | Extremely bad |

### Judgment:

| (Average score) | (Judgment) |
|---|---|
| 5.0 or more | AAA (Excellent) |
| 3.5 or more and less than 5.0 | BBB (Good) |
| 1.5 or more and less than 3.5 | CCC (Fair) |
| Less than 1.5 | DDD (Bad) |

### (Evaluation Method 2)

Among the above evaluation items, the storage stability (d) was evaluated by naked eye observation of a water-containing powdery face powder stored in a sealed airtight container at 50° C. for one month.

| (Evaluation) | (Judgment) |
|---|---|
| Powder is loose and fluid | AAA |
| Powder has a uniform particle size and is fluid | BBB |
| Powder is partly agglomerated | CCC |
| Powder is entirely agglomerated | DDD |

### (Results)

As is clear from Table 1, the Present Invention Preparations 1 to 6 had excellent storage stability, did not impart a powdery or frictional feel, but imparted an excellent fresh feeling upon use, and exhibited excellent applicability without caking or streaking. In addition, the cosmetic preparations showed excellent makeup retention by retaining a transparent feel without darkening over time.

In contrast, Comparative Preparation 1 which contained only a small amount of the component (a), did not exhibit satisfactory storage stability. Comparative Preparation 2, to which a large amount of the component (a) was added, failed to particularly impart a fresh feeling upon use. This preparation was thus unsatisfactory in respect of a fresh feel upon use. Comparative Preparation 3, to which a large amount of the component (b) was added, also failed to impart a fresh feeling upon use. Comparative Preparation 4, which did not contain the component (b), streaking or caking easily occurred during application, darkened with time, and did not retain a transparent feel. Comparative Preparation 5, to which organic spherical particles were added instead of the component (b), did not show satisfactory results in the same items.

### Example 2

### Water-Containing Powdery Eye Color

| (Components) | (%) |
|---|---|
| 1. Glycerin | 1.0 |
| 2. 1,3-Butylene glycol | 10.0 |
| 3. Methyl p-hydroxybenzoate | 0.2 |
| 4. Purified water | 70.0 |
| 5. Hydrophobized silicic anhydride*¹ | 2.5 |
| 6. Plate-like polystyrene powder*⁷ | 6.0 |
| 7. Silicone-treated mica titanium*³ | 5.0 |
| 8. Silicone-treated red No. 226*³ | 0.3 |
| 9. Fluorine-treated talc*⁴ | 5.0 |

*⁷Plate-like particles obtained by pulverizing polystyrene into a thin film (average particle size: 30 μm, average particle thickness: 2 μm, bulk specific gravity: 12 ml/g)

### (Method of Preparation)

A. Components 1 to 4 were mixed and melted at room temperature.
B. A component 5 was added to A and the mixture was blended using a Henschel mixer to obtain a water-containing powder.
C. Components 6 to 9 were added to B and further blended to obtain an eye color.

The eye color of the present invention had good storage stability, did not impart a powdery or frictional feel when used, but imparted an excellent fresh feel upon use, as well as excellent and uniform applicability without caking or streaking. The product also exhibited excellent makeup retention by retaining a transparent feel without darkening of the cosmetic film.

### Example 3

### Water-Containing Powdery Foundation

| (Components) | (%) |
|---|---|
| 1. Agar | 1.5 |
| 2. Carrageenan | 0.1 |
| 3. Glycerin | 1.0 |
| 4. 1,3-Buthylene glycol | 10.0 |
| 5. Methyl p-hydroxybenzoate | 0.1 |
| 6. Purified water | 64.0 |
| 7. Hydrophobized silicic anhydride*¹ | 2.5 |
| 8. Silicone-treated plate-like polystyrene powder*⁸ | 6.0 |
| 9. Silicone-treated colcothar*³ | 0.2 |
| 10. Silicone-treated yellow iron oxide*³ | 1.5 |
| 11. Silicone-treated black iron oxide*³ | 0.1 |
| 12. Fluorine-treated titanium*⁴ | 7.0 |
| 13. Fluorine-treated sericite*⁴ | 4.0 |
| 14. Fluorine-treated talc*⁴ | 2.0 |

*⁸Plate-like particles of *⁷ treated with 3% methyl polysiloxane

### (Method of Preparation)

A. Components 1 to 6 were heated and melted at 85° C., cooled to solidify, and pulverized at −80° C. to obtain an aqueous gel particle (140 to 250 μm).
B. A component 7 was added to A and the mixture was blended using a Henschel mixer to obtain a hydrous powder.
C. Components 8 to 14 were blended B was added thereto with blending to obtain a foundation.

The foundation of the present invention had good storage stability, did not impart a powdery or frictional feel when used, but imparted an excellent fresh feeling upon use, as well as excellent and uniform applicability without caking or streaking. The product also exhibited excellent makeup retention by retaining a transparent feel without darkening of the cosmetic film.

1. A water-containing powdery cosmetic preparation comprising:
(a) 0.1 to 7% by mass of hydrophobic particles having a surface area of at least 80 m²/g;
(b) 0.5 to 30% by mass of plate-like particles comprising a polymer obtained by polymerizing or copolymerizing one or more types of monomers selected from the group consisting of styrenes, methacrylates, and acrylates; and
(c) 50 to 90% by mass of an aqueous component;
wherein the water-containing powdery composition is liquefied by application with rubbing.

2. The water-containing powdery cosmetic preparation according to claim 1, wherein the plate-like particles (b) have a thickness of 0.1 to 20 μm, an average particle size of 5 to 60 μm, and an aspect ratio of at least 10.

3. The water-containing powdery cosmetic preparation according to claim 1, wherein the component (c) comprises an aqueous gelling agent so as to form an aqueous gel.

4. The water-containing powdery cosmetic preparation according to claim 3, wherein the component (c) consists of aqueous gel particles having an average particle size of 1 to 300 μm.

5. The water-containing powdery cosmetic preparation according to claim 1, further comprising:
(d) 0.3 to 5% by mass of an oily component.

6. The water-containing powdery cosmetic preparation according to claim 3, further comprising:
(d) 0.3 to 5% by mass of an oily component.
